(19) United States
(12) Patent Application Publication
(10) Pub. No.: US 2026/0272043 A1
(43) Pub. Date: Sep. 17, 2026

(54) VAPORIZER DEVICE

(71) Applicant: KICNIC LLC, Madisonville, LA (US)

(72) Inventor: Bennett Magee, Madisonville, LA (US)

(73) Assignee: KICNIC LLC, Madisonville, LA (US)

(21) Appl. No.: 19/563,830

(22) Filed: Mar. 11, 2026

Related U.S. Application Data
(60) Provisional application No. 63/770,609, filed on Mar. 12, 2025.

(51) Int. Cl. A24F 40/53; A24F 40/10; A24F 40/42; A24F 40/57; A24F 40/60; A24F 40/65; A61M 15/06

(52) U.S. Cl. CPC A24F 40/53; A24F 40/10; A24F 40/42; A24F 40/57; A24F 40/60; A24F 40/65; A61M 15/06

(57) ABSTRACT

Apparatus and associated methods relate to a vaporizer device including a logic board operatively coupled to a data store storing a set of operations executable by the logic board, including predetermined tier data defining a plurality of usage tiers and tier-transition operations. Each of the plurality of usage tiers may, for example, include an inhalation limit permitted within a corresponding time period. Additionally, each of the plurality of usage tiers may, for example, include a predetermined time interval during which the logic board prevents operation of the vaporizer device. The logic board may, for example, update the current usage tier to a different usage tier by evaluating monitored inhalation activity and selecting the different usage tier based on whether the monitored inhalation activity satisfies a predetermined tier-transition criterion. Various embodiments may advantageously guide gradual reduction of use of a vaporizer device through adaptive usage tiers.

### CROSS-REFERENCE TO RELATED APPLICATIONS

This application is a non-provisional application and claims the benefit of U.S. application Ser. No. 63/770,609, titled “VAPORIZER DEVICE,” filed by Bennett Magee on Mar. 12, 2025.

This application incorporates the entire contents of the foregoing application(s) herein by reference.

### TECHNICAL FIELD

Various embodiments relate generally to vaporizers.

### BACKGROUND

In some industries, scheduling reminders can be useful for helping users complete routine or time-sensitive tasks. Certain applications provide automated reminders that prompt users to perform specific actions at designated times throughout the day. These reminders can help improve consistency, organization, and adherence to schedules.

### SUMMARY

Apparatus and associated methods relate to a vaporizer device including a logic board operatively coupled to a data store storing a set of operations executable by the logic board, including predetermined tier data defining a plurality of usage tiers and tier-transition operations. Each of the plurality of usage tiers may, for example, include an inhalation limit permitted within a corresponding time period. Additionally, each of the plurality of usage tiers may, for example, include a predetermined time interval during which the logic board prevents operation of the vaporizer device. The logic board may, for example, update the current usage tier to a different usage tier by evaluating monitored inhalation activity and selecting the different usage tier based on whether the monitored inhalation activity satisfies a predetermined tier-transition criterion. Various embodiments may advantageously guide gradual reduction of use of a vaporizer device through adaptive usage tiers.

Various embodiments may achieve one or more advantages. For example, some embodiments may, for example, advantageously enable adaptive, tier-based control of device operation. Some implementations may, for example, advantageously dynamically exclude outdated inhalation events from control decisions. Some embodiments may, for example, advantageously implement one-way tier progression logic.

For example, some embodiments may advantageously limit the usage of the vaporize device. Limiting the usage of the vaporizer device may, for example, advantageously assist user in quelling their smoking addiction by initiating timeouts when the user exceeds a certain threshold of usage on the device. This may, for example, advantageously prevent the user from over-consuming nicotine. In some implementations, the vaporizer device may implement timeout logic that advantageously lockouts the vaporizer device when a certain number of uses are exceeded or when a session duration is exceeded. The lockout may, for example, advantageously prevent the user from using the vaporizer device for a certain period of time. This may, for example, advantageously condition the user to abstain from using the vaporizer device.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary vaporizer device employed in an illustrative use-case scenario.

FIG. 2 is a flowchart illustrating an exemplary session-based timeout method of a vaporizer device.

FIG. 3 is a flowchart illustrating an exemplary rolling timer-based timeout method of a vaporizer device.

FIG. 4 depicts an exemplary vaporizer device.

FIG. 5 depicts an exemplary vaporizer device.

FIG. 6 depicts an exemplary vaporizer device.

FIG. 7 depicts an exemplary block diagram of a vaporizer device.

FIG. 8 depicts an exemplary flowchart of a method of operations performed by a processor of a vaporizer device to enforce a lockout duration period.

FIG. 9 depicts an exemplary flowchart of a method of operations performed by a processor of a vaporizer device to manage the transition of usage tiers.

Like reference symbols in the various drawings indicate like elements.

### DETAILED DESCRIPTION OF ILLUSTRATIVE EMBODIMENTS

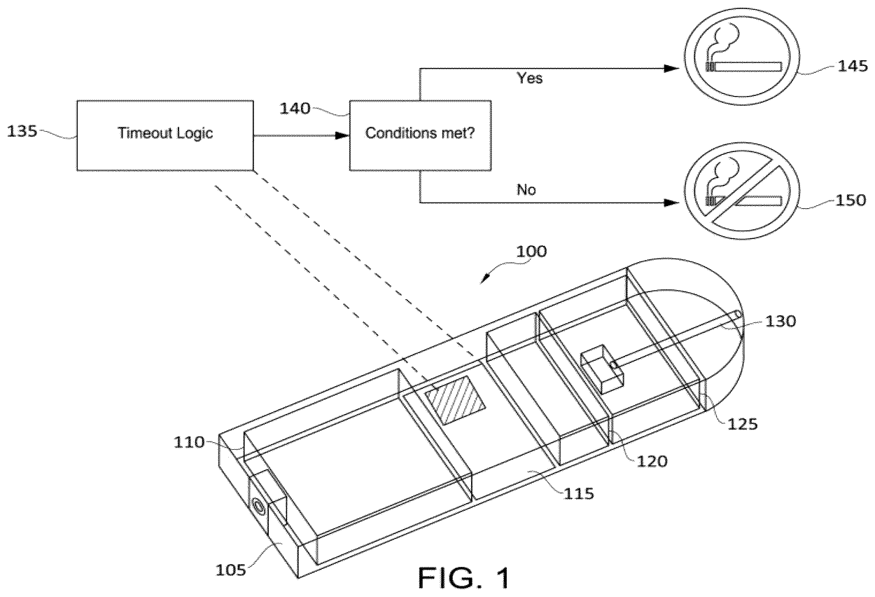

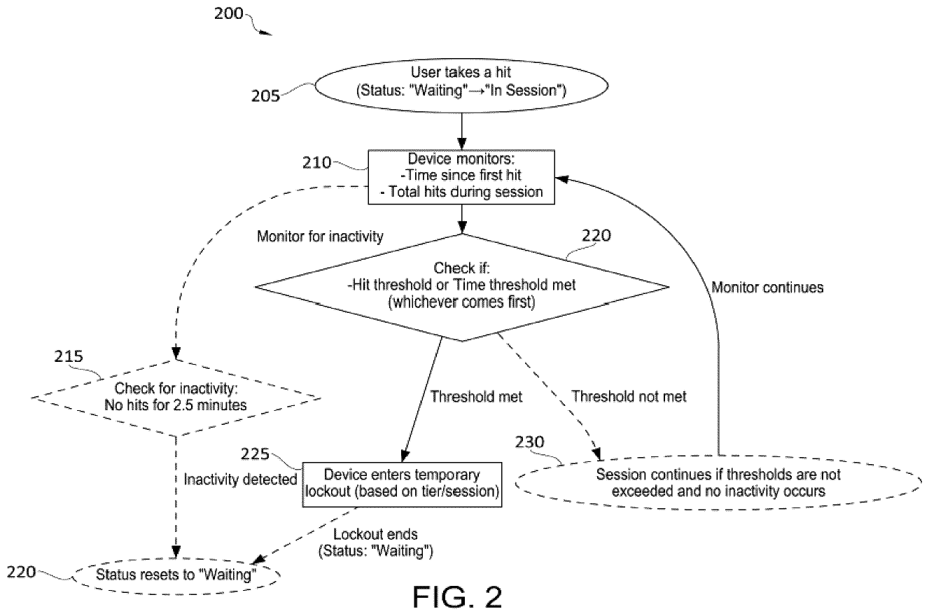

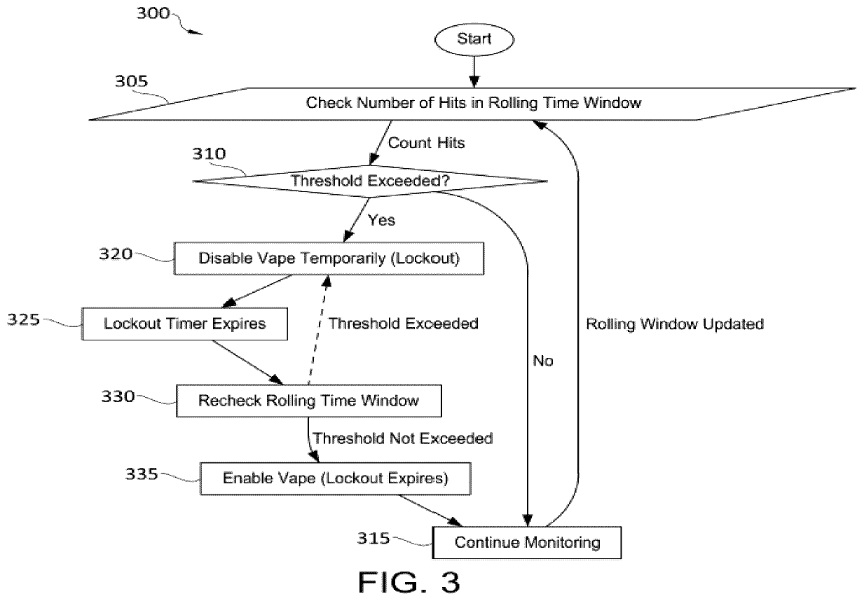

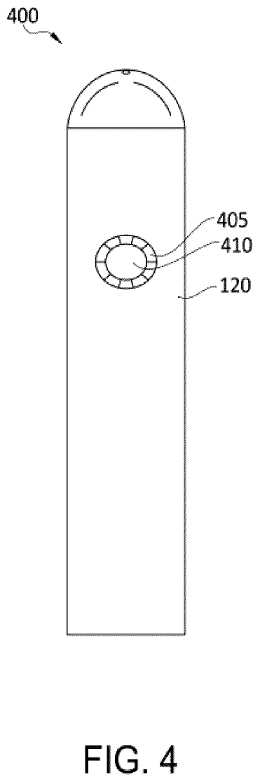

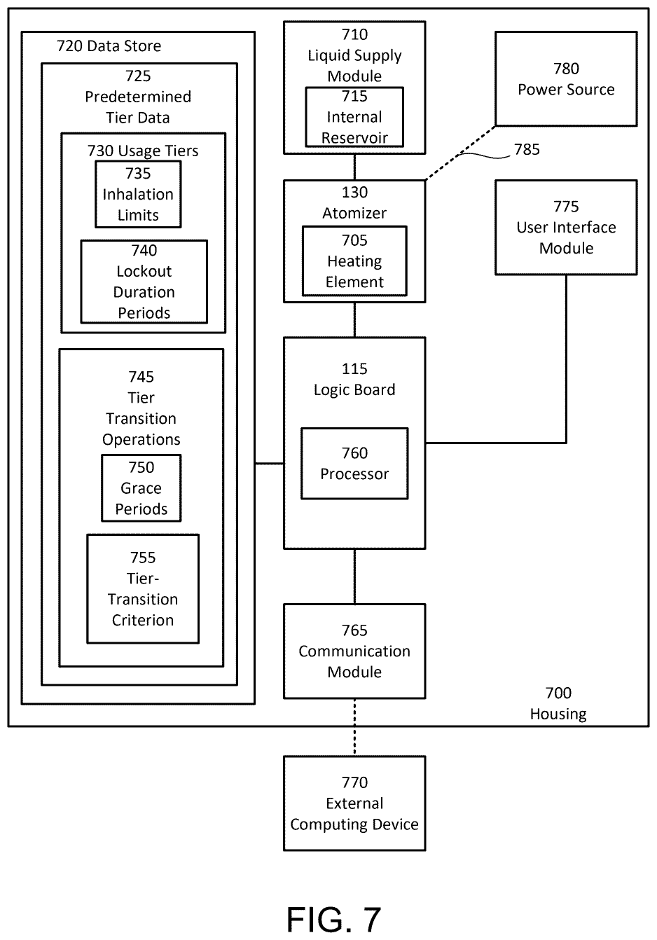

To aid understanding, this document is organized as follows. First, to help introduce discussion of various embodiments, an exemplary vaporizer device is introduced with reference to FIG. 1. Second, that introduction leads into a description with reference to FIGS. 2-3 of some exemplary embodiments of methods utilized by an exemplary vaporizer device. Third, with reference to FIGS. 4-6, the discussion turns to exemplary embodiments that illustrate a vaporizer device. Fourth, with reference to FIGS. 7-9, the document discuses an exemplary block diagram of a vaporizer device and methods of operations performed by a processor of a vaporizer device. Finally, the document discusses further embodiments, exemplary applications and aspects relating to a vaporizer device.

FIG. 1 depicts an exemplary vaporizer device employed in an illustrative use-case scenario. A vaporizer device 100 includes a housing 105, a battery 110, a logic board 115, a user interface module 120, a reservoir 125, and an atomizer 130. The battery 110, the logic board 115, the user interface module 120, the reservoir 125, and the atomizer 130 may, for example, be operably coupled. In some implementations, the logic board 115 may be configured to control operational features in the vaporizer device 100. The logic board 115 may, for example, be configured to implement timeout logic 135 to determine the operation conditions of the vaporizer device 100. In some examples, if conditions 140 are met, then the vaporizer device 100 may be operated 145. If the conditions 140 are not met, then the vaporizer device 100 may not be operated 150. In various implementations, the vaporizer device 100 may include the logic board 115 configured to operate using the timeout logic 135.

In some implementations, the battery 110 may be configured to power the logic board 115, the user interface module 120, and the atomizer 130. For example, the battery 110 may be electrically coupled to the logic board 115, the user interface module 120, and the atomizer 130. In some examples, the battery 110 may be equipped with a charging port. The charging port may, for example, recharge the battery 110.

In various implementations, the logic board 115 may be configured to monitor the activity of the vaporizer device 100. The logic board 115 may, for example, be configured to measure each of the uses, or hits, of the vaporizer device 100. For example, the logic board 115 may initiate a session when a hit is taken by a user. In the session, the logic board 115 may, for example, measure the total number of hits taken by the user. If a user hits a hit threshold, then the logic board 115 may be configured to initiate a lockout of the vaporizer device 100. The lockout may, for example, prevent a user from operating the device. The lockout may, for example, advantageously encourage the user to only take a certain number of hits. For example, the lockout may limit excessive consumption over a session.

In various embodiments, the logic board 115 may initiate a temporary lockout when a hit threshold is exceeded. The temporary lockout may, for example, be configured to operate on a timer. For example, when the timer expires, the logic board 115 may be configured to disable the lockout. In some implementations, the timer may advantageously condition the user of the vaporizer device 100 to abstain from usage.

In some embodiments, the atomizer 130 may be configured to vaporize liquid stored in the reservoir 125. The atomizer 130 may, for example, include a heating element. For example, the heating element may convert the liquid stored in the reservoir 125 into an inhalable vapor.

In some embodiments, the vaporizer device 100 may include a tiered timeout system. The tiered timeout system may, for example, be configured to progressively reduce inhalation limits and extend timeout durations. For example, the vaporizer device 100 may include a tier 5. Tier 5may, for example, allow five inhalations within a 5-minute period, followed by a 5-minute timeout, while a Tier 2 may allow only two inhalations within a 10-minute period, with a 10-minute timeout. In some examples, each tier may correspond to a different group of settings operating on the logic board 115. For example, the logic board 115 may be configured to adjust the tier of the user based on the user's activity. In various embodiments, each tier may correspond to a disposable vaporizer device 100. For example, tier 1-5 disposable vaporizer devices 100 may be preconfigured with timeout parameters. The tiers may, for example, advantageously provide goals and standards for the user to achieve. By reaching different tiers, the user may, for example, be advantageously encouraged to keep abstaining from usage of the vaporizer device 100.

In some implementations, the customization options may be provided by the vaporizer device to tailor the tier settings of the vaporizer device to individual cessation goals. Users may, for example, select vaporizer devices preconfigured for specific tiers or, in some embodiments, adjust timeout durations and inhalation limits through the user interface.

FIG. 2 is a flowchart illustrating an exemplary session-based timeout method of a vaporizer device. The method 200 may, for example, be performed by the logic board 115. In this example method, a user takes a hit of the vaporizer device, initiating a session, and changing the status of the device from “waiting” to “in session” in step 205. A user's hit of the vaporizer device operating in the “waiting” status may, for example, change the status to “in session.” This hit may, for example, be referred to as the initial hit. Monitoring the time since the initial hit and the total number of hits during the session occurs in step 210. In some examples, a determination is made if there is activity after the initial hit. A check may, for example, be made if there is no activity after the initial hit after a period of time (e.g., 2.5 minutes) in step 215. In some implementations, the status may be reset to “waiting” after the period of time has lapsed in step 220.

In some implementations, a determination is made if there is activity after the initial hit. If there is activity after the initial hit, a check may, for example, be made if a threshold is met. The threshold may, for example, be the number of hits taken by the user. The threshold may, for example, be the time duration of the session. In some examples, the threshold may be the number of hits taken by the user and the time duration of the session. For example, if either the number of hits taken threshold or the duration of the session threshold is exceeded, the threshold may be determined as exceeded.

In various implementations, if the threshold is met, the device may enter a temporary lockout in step 225. The temporary lockout may, for example, be based on the tier/session. When the temporary lockout ends, the status may be reset to “waiting” in step 220.

In some embodiments, if the threshold is not met, the session continues and no inactivity occurs in step 230. The session may, for example, continue to be monitored.

In various embodiments, the method 200 may advantageously numerically limit the usage of the vaporizer device 100. For example, the number of hits required to exceed a threshold may advantageously affect the tendency for the user to utilize the vaporizer device 100. The user may, for example, advantageously take less hits and/or reduce their hit frequency.

FIG. 3 is a flowchart illustrating an exemplary rolling timer-based timeout method of a vaporizer device. The method 300 may, for example, be performed by the logic board 115. In this example method, the number of hits in a rolling time window is checked in step 305. The number of hits during the rolling time window may, for example, be limited. If the threshold is not exceeded in step 310, then the number of hits may, for example, continue to be monitored in step 315. If the threshold is exceeded in step 310, then the vaporizer device may be temporarily disabled in a lockout in step 320. The lockout may, for example, be configured to be set to a lockout timer. In some implementations, the lockout timer may expire in step 325. Upon expiration of the timer, the rolling time window may, in some examples, be rechecked to determine if the threshold has been exceeded in step 330. In some embodiments, if the threshold has been exceeded, then the vaporizer device may be temporarily disabled in a lockout in step 320. If the threshold has not been exceeded, then the vaporizer device may, for example, be enabled and the lockout expires in step 335. The monitoring may, in some examples, be continued in step 315. The rolling window may, for example, be configured to be updated with the continued monitoring.

In various embodiments, the method 300 may advantageously limit the user during a particular amount of time. This may, for example, advantageously limit the number of hits of the device 100 for users that smoke frequently during small periods of time. For example, some users may use the vaporizer device 100 during times of stress. These users may, for example, advantageously benefit from the method 300 as it may limit their usage during stressful times.

FIG. 4 depicts an exemplary vaporizer device 400. The user interface module 120 includes indicators 405 and a status check button 410. The indicators 405 may, for example, be LED indicators. In some examples, the indicators 405 may be configured to provide feedback. For example, the indicators 405 may flash to signal the user. The indicators 405 may, for example, advantageously be configured to indicate timeout status. This indication, for example, provide a visual indication that the vaporizer device 100 may be disabled or enabled.

In some implementations, the status check button 410 may be configured to display statuses using the indicators 405. Upon pressing the status check button 410, the user may, for example, be able to determine whether the vaporizer device 100 may be enabled or disabled. For example, pressing the status check button 410 may display a pattern on the indicators 405. The pattern may, for example, advantageously correspond to a status of the vaporizer device 100. For example, the pattern may correspond to a status indicating that the vaporizer device 100 is in a timeout.

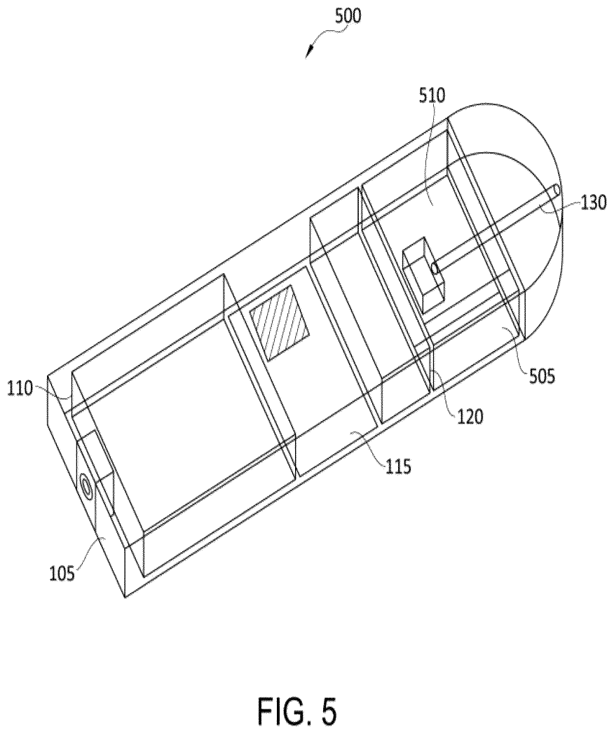

FIG. 5 depicts an exemplary vaporizer device 500. Vaporizer device 500 includes a first reservoir 505 and a second reservoir 510. The reservoirs 505, 510 may, for example, advantageously allow two different liquids to be stored in the vaporizer device 500. In some implementations, the reservoirs 505, 510 may be configured to utilize the same atomizer 130. The reservoirs 505, 510 may, for example, be fluidly coupled to the atomizer 130. This may, for example, advantageously allow the atomizer 130 to vaporize liquid from the reservoirs 505, 510. In some examples, the reservoirs 505, 510 may be coupled to different atomizers. This may, for example, advantageously prevent cross-contamination between the fluids of the reservoirs 505, 510. In some implementations, one of the reservoirs may include a nicotine-infused e-liquid. The other reservoir may, for example, include a nicotine-free e-liquid. The vaporizer device 500 may, for example, be configured to automatically transition from the nicotine reservoir to the nicotine-free reservoir when a threshold is exceeded. For example, this configuration may advantageously assist the user in maintaining the sensory experience associated with using a vaporizer device while limiting further nicotine intake.

In some implementations, the transitioning logic may be controlled by the logic board 115 of the vaporizer device 500. The transitioning logic may, for example, continuously monitor the usage of the vaporizer device 500. This usage monitoring, in some examples, may enforce restrictions on the usage of the vaporizer device without requiring user intervention.

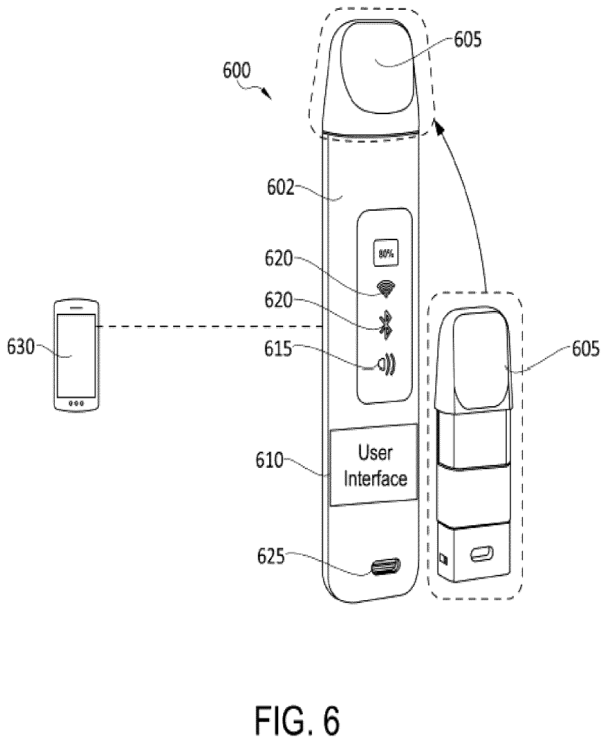

FIG. 6 depicts an exemplary vaporizer device 600. The vaporizer device may, for example, include a housing 602. The housing 602 may define an exterior enclosure of the vaporizer device 600. The housing 602 may, for example, be configured to support, contain, and protect internal components of the vaporizer device. The housing 602 may, for example, be formed as a single-piece structure. The housing 602 may, for example, be formed as multiple portions coupled together.

In some embodiments, the housing 602 may have an elongated and contoured shape configured to be held comfortably by a user during operation. The housing 602 may, for example, include a substantially cylindrical shape. The housing 602 may, for example, include a substantially rectangular shape. The housing 602 may include rounded edges.. The housing 602 may include tapered portions. The housing 602 may include textured exterior surfaces to improve ergonomics, grip, and user comfort. In some implementations, the housing 602 may be dimensioned to be portable and pocket-sized.

The housing 602 may, for example, be fabricated from one or more materials including, but not limited to plastics. The housing 602 may, for example, be fabricated from one or more materials including, but not limited to polymers. The housing 602 may, for example, be fabricated from one or more materials including, but not limited to metals. The housing 602 may, for example, be fabricated from one or more materials including, but not limited to composite materials.

The vaporizer device 600 may, for example, include a liquid supply module 605. The liquid supply module 605 may for example, include an integrated internal reservoir configured to store a liquid. The liquid supply module 605 may, for example, include a replaceable pod. The replaceable pod may, for example, removably couple the vaporizer device 600. For example, the housing 602 may include a cavity configured to receive the liquid supply module 605 such that insertion of the liquid supply module 605 into the cavity fluidly couples the atomizer 130 to the liquid supply module 605. The liquid supply module 605 may, for example, be configured to operatively couple the atomizer 130 such that the liquid supply module 605 is configured to supply liquid to the atomizer 130 when the internal reservoir operatively couples the atomizer.

The vaporizer device 600 may, for example, include a user interface 610. The user interface 610 may, for example, be disposed on an exterior surface of the housing 602. The user interface 610 may, for example, be configured to provide perceptible feedback to a user regarding an operational state of the vaporizer device 600. In some embodiments, the user interface 610 may include one or more visual indicators. The one or more visual indicators may, for example, include light-emitting elements configured to illuminate, change color, or flash. In some embodiments, the user interface 610 may include a display screen configured to present information to the user. In some embodiments, the user interface 610 may include a haptic output 615 configured to generate tactile feedback perceptible to the user. For example, the haptic output 615 may include vibrations of the vaporizer device 600.

The user interface 610 may be configured to convey information. The information may, for example, include usage tier status. The information may, for example, include usage lockout status. The information may, for example, include usage general device state. In some implementations, the user interface 610 may be configured to operate automatically. In some implementations, the user interface 610 may be configured to operate in response to user interaction with the vaporizer device 600. The user interface 610 may, for example, be implemented as a single interface element. The user interface 610 may, for example, be implemented as a combination of multiple interface elements integrated into the housing 602.

In some embodiments, the user interface 610 may be configured to receive user input. The user input may, for example, advantageously permit a user to customize one or more parameters associated with usage tiers. For example, the one or more parameters may include inhalation limits. For example, the one or more parameters may include lockout durations. For example, the one or more parameters may include tier-transition criteria. In some implementations, the user interface 610 may be configured to enable a user to initiate, delay, or acknowledge a transition between usage tiers. In some embodiments, the user input received via the user interface 610 may permit temporary override of tier-transitioning functions.

The vaporizer device 600 may, for example, include a communication module 620. The communication module 620 may, for example, be configured to communicably couple an external computing device 630. For example, the communication module 620 may, wirelessly couple the external computing device 630. For example, the communication module 620 may, couple the external computing device 630 via a wire. For example, the communication module 620 may, couple the external computing device 630 via a mobile application downloaded on the external device 630, such that the mobile application is configured to communicably couple the vaporizer device 600 to the external device, advantageously enabling a user to manage operations of the vaporizer device 600 via the mobile application.

The communication module 620 may, for example, operatively couple the vaporizer device 600 to the external computing device 630 via WIFI. The communication module 620 may, for example, operatively couple the vaporizer device 600 to the external computing device 630 via Bluetooth. The communication module 620 may, for example, operatively couple the vaporizer device 600 to the external computing device 630 via near-field communication (NFC). The communication module 620 may, for example, operatively couple the vaporizer device 600 to the external computing device 630 via a cellular communication protocol. The communication module 620 may, for example, operatively couple the vaporizer device 600 to the external computing device 630 via a wired data interface. The communication module 620 may, for example, operatively couple the vaporizer device 600 to the external computing device 630 via a proprietary short-range wireless protocol.

In some implementations, the communication module 620 may support bidirectional data exchange between the vaporizer device 600 and the external computing device 630, including transmission of usage data, tier data, and device status information, and reception of configuration data, control parameters, or software updates associated with operation of the vaporizer device 600.

The vaporizer device 600 may, for example, include a charging port 625. The charging port 625 may, for example, be configured to electrically couple a charger, such that electrically coupling the charger to the charging port 625 transmit electricity to the vaporizer device's 600 power source.

FIG. 7 depicts an exemplary block diagram of a vaporizer device (e.g., vaporizer device 100, 400, 500, and 600). The vaporizer device in FIG. 7 may, for example, include a housing 700. The housing 700 may, for example, define an exterior enclosure of the vaporizer device. The housing 700 may, for example, be configured to support, contain, and protect internal and external components of the vaporizer device.

The vaporizer device may, for example, include an atomizer 130 disposed within the housing 700. The atomizer 130 may, for example, be configured to vaporize a liquid to generate an inhalable vapor. For example, the atomizer 130 may include a heating element 705. The heating element 705 may, for example, include a resistive metal wire heating coil. The heating element 705 may, for example, include a metal heating strip. The heating element 705 may, for example, include a mesh heating element formed from a conductive metal. The heating element 705 may, for example, include a ceramic heating element embedded with a resistive conductor. The heating element 705 may, for example, include a porous ceramic heater. The heating element 705 may, for example, include a composite heating element combining metal and ceramic materials.

The vaporizer device may, for example, include a liquid supply module 710. The liquid supply module 710 may, for example, include an internal reservoir 715. The internal reservoir 715 may, for example, be configured to store liquid. The internal reservoir 715 may, for example, be configured to supply a liquid to the atomizer when the liquid supply module 710 operatively couples the atomizer 130.

The liquid supply module 710 may, for example, operatively couple the atomizer 130. For example, the liquid supply module 710 may, for example, operatively coupled the atomizer 130 via a fluid pathway that places an internal reservoir 715 of the liquid supply module 710 in fluid communication with the atomizer 130. The liquid supply module 710 may, for example, operatively couple the atomizer 130 via a removable interface that establishes fluid communication between an internal reservoir 715 of the liquid supply module 710 and the atomizer 130 when the liquid supply module 710 is installed in the housing 700.

A data store 720 may, for example, be disposed within the housing 700 and operatively coupled to the logic board 115. The data store 720 may, for example, include a program of instruction. For example, the data store 720 may include predetermined tier data 725. The predetermined tier data 725 may, for example, include usage tiers 730. The usage tiers 730 may, for example, include a plurality of usage tiers, each with a different inhalation limit 735 and lockout duration period 740. The inhalation limit 735 may, for example, include a maximum number of inhalation events permitted within a corresponding time period (e.g., from 1 inhalation event to millions of inhalation events over a time period ranging from fractions of a second to multiple years). An inhalation event may, for example, include a detected instance of user-initiated airflow through the atomizer 130. For example, the inhalation event may be detected by detecting the activation of the heating element 705. The lockout duration period 740 may, for example, include a predetermined time interval during which the atomizer 130 is inhibited such that the vaporizer device is temporarily prevented from generating vapor.

A lockout duration period may, for example, include a predetermined time interval during which a power path 785 between a power source 780 and the atomizer 130 is interrupted. For example, the interruption may occur by the logic board 115 electrically decoupling the atomizer 130 from the power source 780.

The power source 780 may, for example, include a rechargeable battery. The power source 780 may, for example, include a replaceable battery. The power source 780 may, for example, include a supercapacitor. The power source 780 may, for example, include a wired external power supply.

The predetermined tier data 725 may, for example, include tier transition operations 745. The tier transition operations 745 may, for example, be configured to manage how the vaporizer device transitions between usage tiers 730 based on monitored inhalation activity relative to one or more tier transition criteria 755. For example, the tier transition operations 745 may evaluate monitored inhalation events over a defined time interval, compare the evaluated inhalation activity to tier-specific transition criteria stored in the predetermined tier data 725, and update a current usage tier 730 to a different usage tier based on the comparison.

The tier transition criteria 755 may, for example, include a comparison of monitored inhalation activity to one or more tier-specific threshold values. For example, the tier transition criteria 755 may, for example, include determining a cumulative inhalation count within a defined time interval. For example, the tier transition criteria 755 may, for example, include determining temporal spacing between successive inhalation events. For example, the tier transition criteria 755 may, for example, include determining a duration of non-use of the vaporizer device. The tier transition criteria 755 may, for example, include evaluating inhalation activity evaluated within a rolling time window. For example, the rolling time window may include a continuously shifting temporal interval that extends backward from a current moment to a predetermined time in the past and encompasses only inhalation events occurring within the temporal interval, such that inhalation events falling outside the interval are excluded as time advances. The tier transition criteria 755 may, for example, include comparison of monitored inhalation activity to tier-specific threshold values. The tier transition criteria 755 may, for example, include comparison of monitored inhalation activity to tier-specific threshold values during a rolling time window.

The tier transition operations 745 may, for example, include grace periods 750. For example, the grace periods 750 may include transitional time intervals during which a current usage tier 730 is updated to a subsequent usage tier while one or more operational limits applied during the grace period correspond to limits associated with a prior usage tier. For example, upon transitioning from a first usage tier to a second usage tier having a lower inhalation allowance, the grace period 750 may permit inhalation events to be evaluated using an inhalation limit of the first usage tier for a defined duration before application of an inhalation limit associated with the second usage tier.

The logic board 115 may, for example, be disposed within the housing 700. The logic board 115 may, for example, communicably couple the atomizer 130. For example, the atomizer 130 may, for example, communicably couple the processor 760. The processor 760 may, for example, operably couple the data store 720. For example, the processor 760 may be configured to execute the program of instructions stored within the data store 720, such that the processor causes operations to be performed to manage operation of the vaporizer device.

A communication module 765 may, for example, be disposed within the housing 700. The communication module 765 may, for example, communicably couple the logic board 115. For example, the communication module 765 may, for example, communicably couple the processor 760. The communication module 765 may, for example, be substantially similar to the communication module 620. For example, the communication module 765 may communicably couple the external computing device 770 and configured to exchange data with the external computing device 770.

The housing 700 may, for example, operatively couple a user interface module 775. The user interface module 775 may, for example, be disposed on the housing 700. The user interface module 775 may, for example, include a visual indicator. For example, the visual indicator may include one or more light-emitting elements configured to convey an operational state of the vaporizer device, such as availability, lockout status, tier level, or remaining usage allowance.

The user interface may, for example, include a display screen. For example, the display screen may include a liquid crystal display (LCD). For example, the display screen may include an organic light-emitting diode (OLED) display. For example, the display screen may include an electronic ink (e-ink) display. For example, the display screen may include a segmented numeric and/or alphanumeric display.

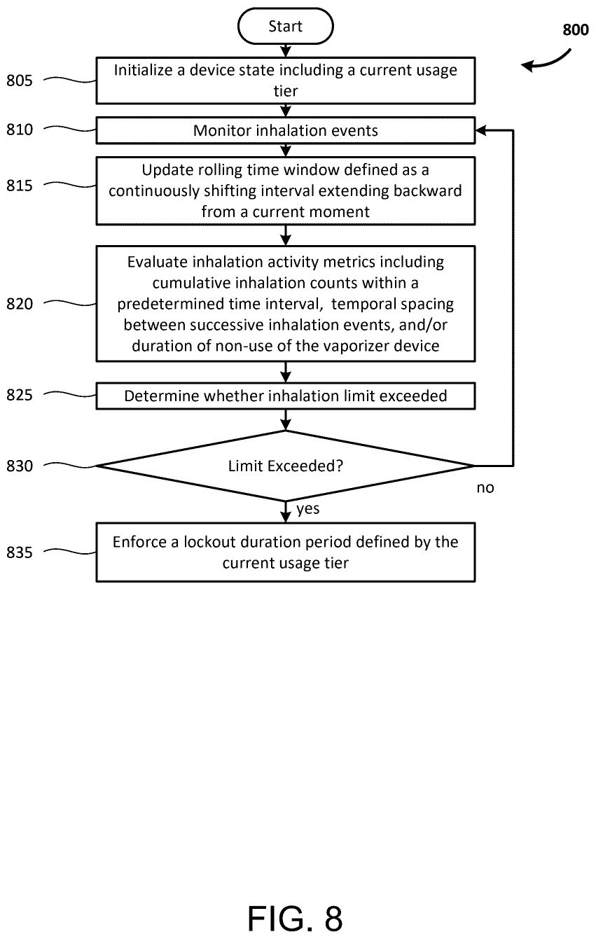

FIG. 8 depicts an exemplary flowchart of a method of operations 800 performed by a processor (e.g., processor 760) of a vaporizer device (e.g., vaporizer device 100, 400, 500, and/or 600) to enforce a lockout duration period (e.g., lockout duration periods 740). In a step 805, the processor initializes a device state including a current usage tier. For example, the processor may retrieve a default usage tier from predetermined tier data (e.g., predetermined tier data 725) stored in a data store (e.g., data store 720). For example, the processor may resume a previously stored usage tier from non-volatile memory. For example, the processor may select an initial usage tier based on a prior usage history associated with the vaporizer device.

In a step 810, the processor may, for example, monitor inhalation events. For example, the processor may receive inhalation event signals from the atomizer and record occurrence data corresponding to detected inhalation events in a data store for subsequent evaluation.

In a step 815, the processor updates the rolling time window defines as a continuously shifting interval extending backward from a current moment. For example, the processor may advance a window boundary based on a current timestamp and exclude inhalation events having timestamps that fall outside the updated interval.

In a step 820, the processor evaluates inhalation activity metrics including cumulative inhalation counts within a predetermined time interval, temporal spacing between successive inhalation events, and/or duration of non-use of the vaporizer device. In a step 825, the processor determines whether an inhalation limit is exceeded. For example, the processor may evaluate inhalation activity by comparing one or more computed inhalation activity metrics to an inhalation limit associated with a current usage tier.

In a step 830, the processor reaches a decision point to decide whether the inhalation limit is exceeded during the predetermined time period. If no, the method of operations 800 proceeds to step 810; if yes, the method of operations 800 proceeds to step 835. In step 835, the processor enforces a lockout duration period defined by the current usage tier. For example, enforcement of the lockout duration period may occur by the processor electrically decoupling the atomizer (e.g., atomizer 130) from the power source (e.g., power source 780).

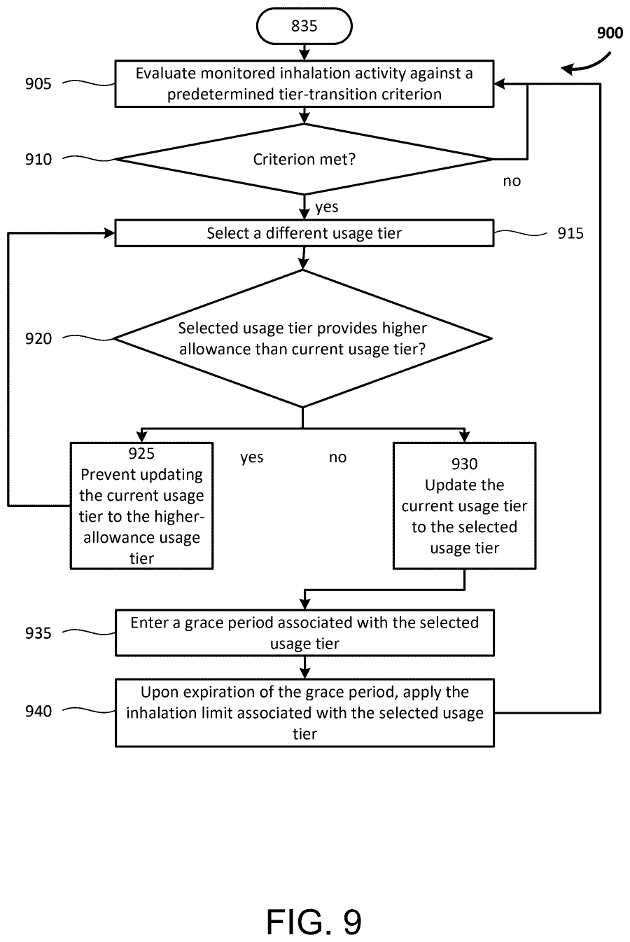

FIG. 9 depicts an exemplary flowchart of a method of operations 900 performed by a processor (e.g., processor 760) of a vaporizer device (e.g., vaporizer device 100, 400, 500, and/or 600) to manage the transition of usage tiers (usage tiers 730). The method of operation 900 may start from step 835 of the method of operations 800. In a step 905, the processor evaluates inhalation activity against a predetermined tier-transition criterion (e.g., tier transition criterion 755). For example, the processor may compare one or more inhalation activity metrics derived from monitored inhalation events to a corresponding threshold associated with the tier-transition criterion.

In a step 910, the processor reaches a decision point where the processor decides whether the tier-transition criterion has been met. If no, the method of operations 900 reverts to step 905; if yes, the method of operation 900 proceeds to step 915.

In a step 915, the processor selects a different usage tier. For example, the processor may identify a subsequent usage tier from a plurality of usage tiers based on satisfaction of the predetermined tier-transition criterion and update the device state to associate operation of the vaporizer device with the selected usage tier.

In a step 920, the processor reaches a decision point where the processor decides whether the selected usage tier process higher allowance than the current usage tier. For example, the processor may compare an inhalation limit associated with the selected usage tier to an inhalation limit associated with the current usage tier to determine whether the selected usage tier permits a greater number of inhalation events within a corresponding time period. If the processor decides yes, the method of operations 900 proceeds to step 925 if the processor decides no, the method oof operations 900 proceeds to step 930.

In step 925, the processor prevents updating the current usage tier to the higher allowance usage tier. For example, the processor may maintain the current usage tier as an active tier when the selected usage tier is determined to permit a greater inhalation allowance than the current usage tier, thereby advantageously enabling one-way progression logic. For example, the processor may apply one-way progression logic that restricts tier transitions to usage tiers having equal or lower inhalation allowances than the current usage tier, thereby maintaining the current usage tier when the selected usage tier would increase an inhalation allowance.

In step 930, the processor updates the current usage tier to the selected usage tier. For example, the processor may the processor may apply operational limits associated with the selected usage tier to subsequent operation of the vaporizer device.

In a step 935, the processor enters a grace period associated with the selected usage tier. For example, the processor may apply operational limits corresponding to a prior usage tier for a defined transitional interval before applying operational limits associated with the selected usage tier.

In a step 940, upon expiration of the grace period, the processor applies the inhalation limit associated with the selected usage tier. For example, the processor may begin evaluating subsequent inhalation events against an inhalation limit defined for the selected usage tier and regulate operation of the vaporizer device in accordance with the applied inhalation limit. After step 940, the method of operations 900 reverts to step 905.

Although various embodiments have been described with reference to the figures, other embodiments are possible.

In some implementations, a vaporizer device may include an ergonomic housing, an atomizer configured to vaporize liquid from a replaceable cartridge, a logic board programmed to monitor user inhalations and enforce timeout intervals, a user interface to display inhalation metrics and timeout progress, and a battery to power the device components.

In various implementations, the atomizer may include a heating element that converts liquid stored in a replaceable cartridge into inhalable vapor. The cartridge may, for example, be designed to be easily replaceable and supports various formulations, including nicotine-based and non-nicotine therapeutic liquids.

In some embodiments, the logic board may be integrated with a flow sensor that detects and records user inhalations. Based on the data, the logic board may, for example, enforce timeout intervals to limit usage after reaching predefined thresholds. The timeout intervals may, for example, be progressively increased in tiers to encourage reduced dependency. For example, the initial tier may permit ten inhalations within 15 minutes, followed by a 30-second timeout, while advanced tiers reduce the allowable inhalations and extend timeout durations.

In various embodiments, the user interface may include an LCD screen or LED indicators to display metrics such as the number of remaining inhalations before a timeout and the progress of ongoing timeouts. In some examples, the battery may be rechargeable via a USB-C port and includes a battery level indicator to notify users of the remaining charge.

In some embodiments, the vaporizer device may include safety features such as overheat protection and automatic shutdown. A temperature sensor may, for example, monitor the atomizer to prevent overheating, ensuring user safety during operation.

In some implementations, customization options may allow users to adjust tier settings and timeout durations via the user interface, tailoring the device to individual cessation goals.

In various implementations, the vaporizer device may include a Bluetooth mechanism or a wireless mechanism that connects to Wi-Fi, enabling the device to pair with a user's smartphone. Through this connection, the user may track their usage of the vaporizer device, such as monitoring compliance with a smoking cessation program, and modify the settings of the device to meet their specific needs. Additionally, the user may, for example, locate a misplaced vaporizer device using their smartphone. In some examples, the vaporizer device may include a speaker or noise-emitting mechanism that can be triggered via Bluetooth or Wi-Fi from the user's smartphone, playing a sound to aid in locating the device. In some embodiments, the vaporizer device may include programmable Bluetooth functionality, allowing the device to serve as a wireless car key, similar to a key fob. This feature may, for example, expand the utility of the device beyond its primary function.

In some implementations, the vaporizer device may incorporate a dynamic timeout mechanism that calculates the length of subsequent timeout periods algorithmically based on the user's prior behavior. For example, the logic board may evaluate metrics such as the number of inhalations during a previous session, the spacing between inhalations, or cumulative usage over a rolling time window. These parameters may, for example, be processed using an adaptive algorithm to determine an appropriate timeout duration, ensuring a personalized and responsive cessation experience.

In various implementations, the vaporizer device may continually monitor and update usage metrics in real-time, employing a rolling time window akin to a dashcam, which continuously records and evaluates recent user behavior. This dynamic approach may, for example, prevent hardcoded limitations and allows for an individualized adjustment of timeout durations based on ongoing and past behavior patterns.

In some embodiments, the vaporizer device may include a dual reservoir system with dynamic switching. In some examples, one reservoir may contain a nicotine-containing e-liquid, while the other may contain a non-nicotine-containing e-liquid. The vaporizer device may, for example, switch to the non-nicotine-containing e-liquid during timeout periods, allowing the user to satisfy their oral fixation without introducing additional nicotine into their system.

In various embodiments, the vaporizer device may dynamically adjust session hit and timeout limits to reward periods of abstinence by the user. For example, if the user abstains from using the vaporizer device for three hours, they may earn a number of “hit credits” (e.g., five) for their next session. This feature may encourage non-use of the vaporizer device while providing a cushion for users who may need additional hits after a period of abstinence.

In some implementations, the vaporizer device may include haptic feedback to effectively communicate the status of the vaporizer device to the user. For example, a vibration may occur when the user takes a hit and has only one hit remaining in the current session. This feature may, for example, advantageously provide a tactile reminder, enhancing user awareness of their usage limits.

In various implementations, the vaporizer device may include a Wi-Fi module that enables the vaporizer device to scan available Wi-Fi access points and adjust its behavior based on detected networks. For instance, when the vaporizer device identifies the “home Wi-Fi” network, the vaporizer device may initiate customized session settings tailored to the user's home environment. Similarly, when the user transitions to their “work Wi-Fi,” the vaporizer device may, for example, activate a unique profile for that location, reinforcing desired habits for each setting.

In various embodiments, the vaporizer device may include an inactivity monitor that resets a session if no inhalations are detected for a predetermined period. This may, for example, ensure that when the user returns to the vaporizer device, it starts a new session rather than resuming the partially completed one.

In some implementations, a logic board may include a central processing unit (CPU), random access memory (RAM), a graphics processing unit (GPU), a chipset, expansion slots, and ports and connectors.

In some embodiments, the housing may include textured a grip surface to prevent slippage during use and is preferably constructed from lightweight, impact-resistant materials. This design may, for example, advantageously ensures that the vaporizer device remains easy to handle, even in conditions where the user's hands may be wet or oily

In various embodiments, to prevent leakage, the atomizer may include a sealed coupling mechanism that secures the replaceable cartridge to the atomizer, ensuring proper alignment for efficient vaporization.

In some implementations, the reservoirs may hold up to 2 milliliters of liquid, making it suitable for extended use. The vaporizer device may, for example, include an integrated wicking system that ensures even delivery of liquid to the heating element, preventing dry hits and maintaining consistent vapor production.

In various implementations, the logic board may control all operational and safety features. The logic board may, for example, be integrated with a flow sensor located within the air intake pathway. The flow sensor may, for example, detect each inhalation and relays this information to the logic board, which tracks the number of inhalations over time and enforces timeout intervals when predefined thresholds are exceeded. For example, the vaporizer device may include a timeout mechanism that may be implemented by the logic board. In some examples, the timeout mechanism implemented by the logic board may be digitally controlled

In some embodiments, during a timeout period, the logic board may deactivate the atomizer by disconnecting the battery from the heating element. This solid-state approach may, for example, advantageously eliminate the need for moving parts, enhancing the vaporizer device's reliability and lifespan.

In various embodiments, the user interface of the vaporizer device provides real-time feedback to users. In one embodiment, the user interface may, for example, includes LED indicators that flash or change color to signal timeout status, remaining inhalations, or battery level. In another embodiment, the vaporizer device may, for example, include an LCD screen to display detailed metrics, such as the number of remaining inhalations before a timeout, the progress of an active timeout, and the battery's charge status.

In some implementations, users may select vaporizer devices preconfigured for specific tiers, such as a Tier 5 vaporizer device that allows five inhalations within a 5-minute period, or adjust timeout durations and inhalation limits through the user interface module. In some examples, users may set personalized intervals, such as changing a Tier 2 timeout from 10 minutes to 8 minutes to better match their preferences. These adjustments may, for example, be made directly on the vaporizer device via an LCD screen or, in another embodiment, through a companion smartphone app that synchronizes with the vaporizer device 100 using Bluetooth or Wi-Fi. Such customization may, for example, empower users to tailor the vaporizer device to their individual smoking cessation needs, improving adherence to their goals.

In various implementations, the battery may be rechargeable. The rechargeable battery may, for example, power all components of the vaporizer device, including the atomizer, the logic board, and the user interface. The battery may, for example, be equipped with a USB-C port for fast charging, advantageously enabling the vaporizer device to achieve a full charge in under one hour. A multi-bar battery level indicator may, for example, advantageously visually communicate the remaining charge to the user, reducing the likelihood of unexpected downtime.

In some embodiments, safety features may be integrated into the vaporizer device to ensure secure and reliable operation. A temperature sensor embedded within the atomizer may, for example, monitor the heating element to prevent overheating. If the temperature sensor detects unsafe operating conditions, the logic board may, for example, temporarily disables the atomizer until the temperature returns to a safe level.

In various embodiments, the vaporizer device may utilize a programmable Bluetooth mechanism, enabling advanced functionality. For example, the vaporizer device may serve as a wireless car key, much like a programmable key fob, advantageously allowing users to unlock or lock their vehicle without the need for a traditional key. Additionally, the Bluetooth functionality enables lost vaporizer device tracking. If the vaporizer device is misplaced, a paired smartphone application may, for example, send a signal to the vaporizer device, activating a sound through an integrated speaker, making it easier to locate. In some examples, this feature may be particularly useful for instances where the vaporizer device falls between couch cushions or be otherwise misplaced. Users may, for example, also use the Bluetooth connection to monitor usage metrics, adjust timeout settings, and receive notifications regarding battery life or inhalation limits. This additional capability may, for example, broaden the utility of the vaporizer device, integrating it with other devices and applications.

In some implementations, the vaporizer device may incorporate a dynamic timeout mechanism. In such embodiment, the logic board may, for example, calculate timeout durations algorithmically, taking into account prior user behavior such as the number of inhalations, the spacing between inhalations, and cumulative usage over a rolling time window. In some examples, the logic board may calculate timeout durations algorithmically based on prior user behavior, such as the number of inhalations, the spacing between inhalations, and cumulative usage over a rolling time window. For example, if a user may take frequent and closely spaced inhalations, the logic board may dynamically increases the timeout period to encourage reduced consumption. This adaptive algorithm may, for example, provide a personalized cessation experience while maintaining the vaporizer device's effectiveness across diverse user behaviors.

In various implementations, the rolling time window functions similarly to a dashcam, continuously analyzing the most recent behavior while discarding older data. For instance, the vaporizer device may evaluate the user's behavior over the past 5 to 10 minutes, amalgamating metrics such as the frequency and spacing of inhalations to determine an appropriate timeout duration. If the user takes frequent, closely spaced inhalations, the vaporizer device may dynamically increases the timeout period to encourage reduced consumption. This approach may, for example, allow the logic board to adapt the timeout mechanism to real-time conditions without relying on static parameters. In some examples, the rolling window evaluates the user's behavior over the past 5 to 10 minutes, amalgamating metrics such as inhalation count and spacing to calculate a responsive timeout duration.

In some embodiments, by eliminating static, hardcoded timeout settings, an adaptive algorithm may, for example, provide a personalized and effective cessation experience, encouraging compliance with smoking cessation goals while maintaining flexibility to address individual needs. In other words, by employing an adaptive algorithm, the vaporizer device may eliminate hardcoded timeout durations, providing a personalized and dynamic cessation experience. This functionality may, for example, advantageously ensure the vaporizer device remains effective across a wide range of user behaviors, encouraging compliance with smoking cessation goals while maintaining flexibility to address individual needs.

In various embodiments, the vaporizer device may be designed for a wide range of applications beyond nicotine cessation. In another embodiment, the vaporizer device may, for example, deliver therapeutic compounds, such as CBD or decongestants, making it suitable for medical or wellness applications. In some examples, this versatility advantageously expands the utility and market potential of the vaporizer device, addressing a broader spectrum of user needs and preferences.

In some implementations, the vaporizer device may be a disposable vape device designed to assist users in reducing nicotine addiction over time. The vaporizer device may, for example, introduce innovative functionality, providing both a healthier alternative and a gradual pathway to reducing dependence.

In accordance with some embodiments, a vaporizer device may include an ergonomic housing; an atomizer configured to vaporize a liquid; a replaceable cartridge removably coupled to the atomizer, the replaceable cartridge having an internal reservoir; a logic board configured to monitor and record user inhalation behavior and enforce a tiered timeout mechanism; a user interface configured to display inhalation limits and timeout progress; and a power source operatively connected to the atomizer and logic board.

In some embodiments, the logic board may be configured to calculate timeout durations algorithmically based on the user inhalation behavior.

In some implementations, the tiered timeout mechanism may, for example, reduce inhalation limits and increases timeout durations progressively.

In various embodiments, the user interface may include an LCD screen configured to display the tier level and remaining inhalation limits.

In some implementations, the vaporizer device may include a Bluetooth module adapted to synchronize with a user's smartphone.

In various implementations, the vaporizer device may include a sound mechanism in communication with the logic board. the Bluetooth module may, for example, be configured to locate the vaporizer device when misplaced using the sound mechanism upon receipt of a signal from the user's smartphone when synchronized

In some implementations, the logic board may dynamically adjust timeout durations based on a rolling time window of the monitored and recorded user inhalation behavior.

In accordance with some embodiments, the vaporizer device may include a programmable wireless communication module configured to enable synchronization with external devices

In accordance with some embodiments, a vaporizer device may include an ergonomic housing; an atomizer configured to vaporize a liquid; a replaceable cartridge removably coupled to the atomizer, the replaceable cartridge having an internal reservoir; a logic board configured to monitor and record user inhalation behavior and enforce a tiered timeout mechanism; a user interface configured to display inhalation limits and timeout progress; and a power source operatively connected to the atomizer and logic board.

In some embodiments, the logic board may be configured to calculate timeout durations algorithmically based on the user inhalation behavior.

In some implementations, the tiered timeout mechanism may, for example, reduce inhalation limits and increases timeout durations progressively.

In various embodiments, the user interface may include an LCD screen configured to display the tier level and remaining inhalation limits.

In some implementations, the vaporizer device may include, for example, a Bluetooth module adapted to synchronize with a user's smartphone.

In various implementations, the vaporizer device may include, for example, a sound mechanism in communication with the logic board. the Bluetooth module may, for example, be configured to locate the vaporizer device when misplaced using the sound mechanism upon receipt of a signal from the user's smartphone when synchronized

In some implementations, the logic board may, for example, dynamically adjust timeout durations based on a rolling time window of the monitored and recorded user inhalation behavior.

In accordance with some embodiments, the vaporizer device may, for example, include a programmable wireless communication module configured to enable synchronization with external devices.

In various embodiments, a vaporizer device may include, for example, an ergonomic housing; an atomizer configured to vaporize a liquid; a replaceable cartridge removably coupled to the atomizer, the replaceable cartridge having an internal reservoir; a logic board configured to monitor user inhalation behavior and enforce a tiered timeout mechanism, the logic board initiates a session upon user inhalation and tracks the duration and frequency of inhalations during the session; a user interface configured to display inhalation limits and timeout progress; a power source operatively connected to the atomizer and logic board; and a dual reservoir system with dynamic switching, one reservoir contains a nicotine-containing e-liquid and the other contains a non-nicotine-containing e-liquid, and the vaporizer device switches to the non-nicotine-containing e-liquid during timeout periods.

In some implementations, the vaporize device may, for example, include a dual reservoir system that may, for example, be configured to switch to the non-nicotine-containing e-liquid if the user attempts to take a hit during a timeout period.

In various implementations, the vaporizer device may include an inactivity monitor that resets the session if no inhalations are detected for a predetermined period.

In some embodiments, the vaporizer device may, for example, include a logic board that dynamically adjusts timeout durations based on a rolling time window of the monitored and recorded user inhalation behavior.

In some implementations, the vaporizer device may, for example, include the logic board that is programmed to award additional inhalation credits for a next session based on periods of non-use, the logic board calculates a number of additional inhalation credits based on the duration of the non-use period.

In various implementations, the vaporizer device may include a haptic feedback mechanism coupled to the logic board, said haptic feedback mechanism configured to provide tactile alerts to the user, including alerts for low battery status and remaining inhalations.

In some embodiments, the vaporizer device may include a logic board having a countdown feature and that is adapted to send a signal to the haptic feedback mechanism to initiate different vibrations indicating the number of remaining inhalations.

In various embodiments, the vaporizer device may include a Wi-Fi module configured to scan available Wi-Fi networks and adjust the vaporizer device's behavior based on detected networks.

In some implementations, the vaporizer device may include a Wi-Fi module adapted to scan for network names, the Wi-Fi module not requiring an active connection to a Wi-Fi network having one of said network names.

In some embodiments, the Wi-Fi module may adjust the session time and hit threshold based on the names of the detected Wi-Fi networks.

In various embodiments, the wireless communication module may be configured to serve as a programmable key fob for a vehicle.

In some implementations, the vaporizer device may, for example, be configured to track usage metrics and display said usage metrics on a user's smartphone.

In various implementations, the vaporizer device may, for example, include a speaker configured to produce a sound for locating the device.

In some embodiments, a method of operating a vaporizer device may include the step of providing a vaporizer device having an ergonomic housing, an atomizer configured to vaporizer a liquid; a replaceable cartridge removably coupled to the atomizer, the replaceable cartridge having an internal reservoir; a logic board configured to monitor and record user inhalation behavior and enforce a tiered timeout mechanism; a user interface configured to display inhalation limits and timeout progress; and a power source operatively connected to the atomizer and logic board.

In various embodiments, a method of operating a vaporizer device including the steps of providing a vaporizer device, detecting an inhalation event using a flow sensor; monitoring inhalation behavior over a rolling time window; calculating a dynamic timeout period based on the inhalation behavior; enforcing the dynamic timeout period by disconnecting the atomizer from a power source; and displaying inhalation limits and timeout progress on a user interface.

In some implementations, a method of operating a vaporizer device including a rolling time window that may, for example, be configured to evaluate the frequency and spacing of inhalations over the past 5 to 10 minutes.

In various implementations, a method of operating a vaporizer including the step of dynamically adjusting the timeout period based on cumulative inhalation counts during the rolling time window.

In some embodiments, a method of operating a vaporizer including the step of synchronizing the vaporizer device with a user's smartphone using a wireless communication module.

In various embodiments, a method of operating a vaporizer including the step of displaying the current tier level and timeout status on an LCD screen.

In some implementations, the wireless communication module may be configured to emit a sound when triggered by the user's smartphone.

In various implementations, the timeout mechanism may be implemented using an adaptive algorithm to encourage compliance with cessation goals.

Although an exemplary system has been described with reference to FIGS. 1-9, other implementations may be deployed in other industrial, scientific, medical, commercial, and/or residential applications.

In various embodiments, some bypass circuits implementations may be controlled in response to signals from analog or digital components, which may be discrete, integrated, or a combination of each. Some embodiments may include programmed, programmable devices, or some combination thereof (e.g., PLAs, PLDs, ASICs, microcontroller, microprocessor), and may include one or more data stores (e.g., cell, register, block, page) that provide single or multi-level digital data storage capability, and which may be volatile, non-volatile, or some combination thereof. Some control functions may be implemented in hardware, software, firmware, or a combination of any of them.

Computer program products may contain a set of instructions that, when executed by a processor device, cause the processor to perform prescribed functions. These functions may be performed in conjunction with controlled devices in operable communication with the processor. Computer program products, which may include software, may be stored in a data store tangibly embedded on a storage medium, such as an electronic, magnetic, or rotating storage device, and may be fixed or removable (e.g., hard disk, floppy disk, thumb drive, CD, DVD).

Although an example of a system, which may be portable, has been described with reference to the above figures, other implementations may be deployed in other processing applications, such as desktop and networked environments.

Temporary auxiliary energy inputs may be received, for example, from chargeable or single use batteries, which may enable use in portable or remote applications. Some embodiments may operate with other DC voltage sources, such as a 9V (nominal) batteries, for example. Alternating current (AC) inputs, which may be provided, for example from a 50/60 Hz power port, or from a portable electric generator, may be received via a rectifier and appropriate scaling. Provision for AC (e.g., sine wave, square wave, triangular wave) inputs may include a line frequency transformer to provide voltage step-up, voltage step-down, and/or isolation.

Although particular features of an architecture have been described, other features may be incorporated to improve performance. For example, caching (e.g., L1, L2, . . . ) techniques may be used. Random access memory may be included, for example, to provide scratch pad memory and or to load executable code or parameter information stored for use during runtime operations. Other hardware and software may be provided to perform operations, such as network or other communications using one or more protocols, wireless (e.g., infrared) communications, stored operational energy and power supplies (e.g., batteries), switching and/or linear power supply circuits, software maintenance (e.g., self-test, upgrades), and the like. One or more communication interfaces may be provided in support of data storage and related operations.

Some systems may be implemented as a computer system that can be used with various implementations. For example, various implementations may include digital circuitry, analog circuitry, computer hardware, firmware, software, or combinations thereof. Apparatus can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Various embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, which may include a single processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and CD-ROM and DVD-ROM disks. The processor and the memory can be supplemented by, or incorporated in, ASICs (application-specific integrated circuits).

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. Various embodiments may be implemented in a computer system that includes a graphical user interface and/or an Internet browser. To provide for interaction with a user, some implementations may be implemented on a computer having a display device. The display device may, for example, include an LED (light-emitting diode) display. In some implementations, a display device may, for example, include a CRT (cathode ray tube). In some implementations, a display device may include, for example, an LCD (liquid crystal display). A display device (e.g., monitor) may, for example, be used for displaying information to the user. Some implementations may, for example, include a keyboard and/or pointing device (e.g., mouse, trackpad, trackball, joystick), such as by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, the computers and networks forming the Internet, or some combination thereof. Other implementations may transport messages by broadcasting to all or substantially all devices that are coupled together by a communication network, for example, by using omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Firewire, ATA/IDE, RS-232, RS-422, RS-485, 802.11 a/b/g, Wi-Fi, Ethernet, IrDA, FDDI (fiber distributed data interface), token-ring networks, multiplexing techniques based on frequency, time, or code division, or some combination thereof. Some implementations may optionally incorporate features such as error checking and correction (ECC) for data integrity, or security measures, such as encryption (e.g., WEP) and password protection.

In various embodiments, the computer system may include Internet of Things (IoT) devices. IoT devices may include objects embedded with electronics, software, sensors, actuators, and network connectivity which enable these objects to collect and exchange data. IoT devices may be in-use with wired or wireless devices by sending data through an interface to another device. IoT devices may collect useful data and then autonomously flow the data between other devices.

Various examples of modules may be implemented using circuitry, including various electronic hardware. By way of example and not limitation, the hardware may include transistors, resistors, capacitors, switches, integrated circuits, other modules, or some combination thereof. In various examples, the modules may include analog logic, digital logic, discrete components, traces and/or memory circuits fabricated on a silicon substrate including various integrated circuits (e.g., FPGAs, ASICs), or some combination thereof. In some embodiments, the module(s) may involve execution of preprogrammed instructions, software executed by a processor, or some combination thereof. For example, various modules may involve both hardware and software.

In some aspects, the techniques described herein relate to a vaporizer device, including: a housing; an atomizer disposed within the housing and configured to vaporize a liquid to generate an inhalable vapor; a liquid supply module including an internal reservoir and configured to operatively couple the atomizer, wherein the internal reservoir is configured to store the liquid and supply the liquid to the atomizer when the internal reservoir operatively couples the atomizer; a data store disposed within the housing and including a program of instructions, the program of instructions including predetermined tier data defining a plurality of usage tiers and tier-transition operations, such that each usage tier of the plurality of usage tiers includes: an inhalation limit including a maximum number of inhalation events permitted within a corresponding predetermined time period; and a lockout duration period including a predetermined time interval during which operation of the atomizer is prevented; and, a logic board disposed within the housing and communicably coupled to the atomizer, wherein the logic board includes a processor operably coupled to the data store such that, when the processor executes the program of instructions, the processor causes operations to be performed to manage operation of the vaporizer device, the operations including: monitor inhalation events of a user; determine, for a current usage tier, whether the monitored inhalation events exceed the inhalation limit of the current usage tier within the corresponding time period; upon determining that the inhalation limit is exceeded, enforce a lockout duration period by disabling the atomizer for the lockout duration period defined by the current usage tier such that the user is locked out from using the vaporizer device during the lockout; and update the current usage tier to a different usage tier of the plurality of usage tiers by executing the tier-transition operations, the tier-transition operations causing the logic board to evaluate monitored inhalation activity of the user and select the different usage tier based on whether the monitored inhalation activity satisfies a predetermined tier-transition criterion.

In some aspects, the techniques described herein relate to a vaporizer device, wherein the liquid supply module further includes a replaceable pod removably coupled to the housing.

In some aspects, the techniques described herein relate to a vaporizer device, wherein the operations further include: maintain one-way progression through the plurality of usage tiers such that, after the current usage tier is updated to a lower-allowance usage tier, prevent updating the current usage tier to a higher-allowance usage tier.

In some aspects, the techniques described herein relate to a vaporizer device, wherein the tier-transition operations further include a grace period associated with a usage tier change, the grace period defining a temporary interval during which the processor updates the current usage tier to a different usage tier of the plurality of usage tiers such that the inhalation limit applied during the grace period corresponds to the inhalation limit of a prior usage tier.

In some aspects, the techniques described herein relate to a vaporizer device, wherein monitoring inhalation events includes evaluating inhalation events within a rolling time window defined as a continuously shifting interval that extends backward from a current moment and includes only inhalation events occurring within the interval, such that inhalation events occurring prior to the interval are excluded as time advances.

In some aspects, the techniques described herein relate to a vaporizer device, further including a user interface module disposed on the housing and including a visual indicator.

In some aspects, the techniques described herein relate to a vaporizer device, further including a user interface module disposed on the housing and including a display screen.

In some aspects, the techniques described herein relate to a vaporizer device, further including a user interface module disposed on the housing and including a haptic output.

In some aspects, the techniques described herein relate to a vaporizer device, further including a communication module communicably coupled to the logic board, the wireless communication module configured to exchange data with an external computing device.

In some aspects, the techniques described herein relate to a vaporizer device, wherein enforcing the lockout duration period includes controlling a power path between a power source and the atomizer to prevent energizing a heating element of the atomizer during the lockout duration period.

In some aspects, the techniques described herein relate to a vaporizer device, wherein the tier-transition criterion includes determining a cumulative inhalation count over a predetermined time interval.

In some aspects, the techniques described herein relate to a vaporizer device, wherein the tier-transition criterion includes evaluating time intervals between successive inhalation events and determining whether to update a usage tier based on the evaluated time intervals.

In some aspects, the techniques described herein relate to a vaporizer device, wherein the tier-transition criterion includes measuring a duration of non-use of the vaporizer device and determining whether to update a usage tier based on the measured duration.

In some aspects, the techniques described herein relate to a computer-implemented method performed by at least one processor to manage operation of a vaporizer device, the method including: monitor inhalation events of a user; determine, for a current usage tier, whether the monitored inhalation events exceed an inhalation limit of the current usage tier within a corresponding time period; upon determining that the inhalation limit is exceeded, enforce a lockout duration period by disabling an atomizer of the vaporizer device for a predetermined lockout duration defined by the current usage tier such that the user is locked out from using the vaporizer device during the lockout; and update the current usage tier to a different usage tier of a plurality of usage tiers by executing tier-transition operations, the tier-transition operations causing a logic board of the vaporizer device to evaluate monitored inhalation activity of the user and select the different usage tier based on whether the monitored inhalation activity satisfies a predetermined tier-transition criterion.

In some aspects, the techniques described herein relate to a computer-implemented method, wherein the tier-transition criterion includes determining a cumulative inhalation count over a predetermined time interval.

In some aspects, the techniques described herein relate to a computer-implemented method, wherein the tier-transition criterion includes evaluating time intervals between successive inhalation events and determining whether to update a usage tier based on the evaluated time intervals.

In some aspects, the techniques described herein relate to a computer-implemented method, wherein the tier-transition criterion includes measuring a duration of non-use of the vaporizer device and determining whether to update a usage tier based on the measured duration.

In some aspects, the techniques described herein relate to a computer program product (CPP) including a program of instructions tangibly embodied on a non-transitory computer readable medium wherein, when the instructions are executed on a processor, the processor causes operations to be performed to manage operation of a vaporizer device, the operations including: monitor inhalation events of a user; determine, for a current usage tier, whether the monitored inhalation events exceed an inhalation limit of the current usage tier within a corresponding time period; upon determining that the inhalation limit is exceeded, enforce a lockout duration period by disabling an atomizer of the vaporizer device for a predetermined lockout duration defined by the current usage tier such that the user is locked out from using the vaporizer device during the lockout; and update the current usage tier to a different usage tier of a plurality of usage tiers by executing tier-transition operations, the tier-transition operations causing a logic board of the vaporizer device to evaluate monitored inhalation activity of the user and select the different usage tier based on whether the monitored inhalation activity satisfies a predetermined tier-transition criterion.

In some aspects, the techniques described herein relate to a computer program product, wherein monitoring inhalation events includes evaluating inhalation events within a rolling time window defined as a continuously shifting interval that extends backward from a current moment and includes only inhalation events occurring within the interval, such that inhalation events occurring prior to the interval are excluded as time advances.

In some aspects, the techniques described herein relate to a computer program product, wherein the operations further include: maintain one-way progression through the plurality of usage tiers such that, after the current usage tier is updated to a lower-allowance usage tier, prevent updating the current usage tier to a higher-allowance usage tier.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A vaporizer device, comprising:
a housing;
an atomizer disposed within the housing and configured to vaporize a liquid to generate an inhalable vapor;
a liquid supply module comprising an internal reservoir and configured to operatively couple the atomizer, wherein the internal reservoir is configured to store the liquid and supply the liquid to the atomizer when the internal reservoir operatively couples the atomizer;
a data store disposed within the housing and comprising a program of instructions, the program of instructions comprising predetermined tier data defining a plurality of usage tiers and tier-transition operations, such that each usage tier of the plurality of usage tiers comprises:
an inhalation limit including a maximum number of inhalation events permitted within a corresponding predetermined time period; and
a lockout duration period comprising a predetermined time interval during which operation of the atomizer is prevented; and,
a logic board disposed within the housing and communicably coupled to the atomizer, wherein the logic board comprises a processor operably coupled to the data store such that, when the processor executes the program of instructions, the processor causes operations to be performed to manage operation of the vaporizer device, the operations comprising:
monitor inhalation events of a user;
determine, for a current usage tier, whether the monitored inhalation events exceed the inhalation limit of the current usage tier within the corresponding time period;
upon determining that the inhalation limit is exceeded, enforce a lockout duration period by disabling the atomizer for the lockout duration period defined by the current usage tier such that the user is locked out from using the vaporizer device during the lockout; and
update the current usage tier to a different usage tier of the plurality of usage tiers by executing the tier-transition operations, the tier-transition operations causing the logic board to evaluate monitored inhalation activity of the user and select the different usage tier based on whether the monitored inhalation activity satisfies a predetermined tier-transition criterion.

2. The vaporizer device of claim 1, wherein the liquid supply module further comprises a replaceable pod removably coupled to the housing.

3. The vaporizer device of claim 1, wherein the operations further comprise:
maintain one-way progression through the plurality of usage tiers such that, after the current usage tier is updated to a lower-allowance usage tier, prevent updating the current usage tier to a higher-allowance usage tier.

4. The vaporizer device of claim 1, wherein the tier-transition operations further comprise a grace period associated with a usage tier change, the grace period defining a temporary interval during which the processor updates the current usage tier to a different usage tier of the plurality of usage tiers such that the inhalation limit applied during the grace period corresponds to the inhalation limit of a prior usage tier.

5. The vaporizer device of claim 1, wherein monitoring inhalation events comprises evaluating inhalation events within a rolling time window defined as a continuously shifting interval that extends backward from a current moment and includes only inhalation events occurring within the interval, such that inhalation events occurring prior to the interval are excluded as time advances.

6. The vaporizer device of claim 1, further comprising a user interface module disposed on the housing and comprising a visual indicator.

7. The vaporizer device of claim 1, further comprising a user interface module disposed on the housing and comprising a display screen.

8. The vaporizer device of claim 1, further comprising a user interface module disposed on the housing and comprising a haptic output.

9. The vaporizer device of claim 1, further comprising a communication module communicably coupled to the logic board, the communication module configured to exchange data with an external computing device.

10. The vaporizer device of claim 1, wherein enforcing the lockout duration period comprises controlling a power path between a power source and the atomizer to prevent energizing a heating element of the atomizer during the lockout duration period.

11. The vaporizer device of claim 1, wherein the tier-transition criterion comprises determining a cumulative inhalation count over a predetermined time interval.

12. The vaporizer device of claim 1, wherein the tier-transition criterion comprises evaluating time intervals between successive inhalation events and determining whether to update a usage tier based on the evaluated time intervals.

13. The vaporizer device of claim 1, wherein the tier-transition criterion comprises measuring a duration of non-use of the vaporizer device and determining whether to update a usage tier based on the measured duration.

14. A computer-implemented method performed by at least one processor to manage operation of a vaporizer device, the method comprising:
monitor inhalation events of a user;
determine, for a current usage tier, whether the monitored inhalation events exceed an inhalation limit of the current usage tier within a corresponding time period;
upon determining that the inhalation limit is exceeded, enforce a lockout duration period by disabling an atomizer of the vaporizer device for a predetermined lockout duration defined by the current usage tier such that the user is locked out from using the vaporizer device during the lockout; and
update the current usage tier to a different usage tier of a plurality of usage tiers by executing tier-transition operations, the tier-transition operations causing a logic board of the vaporizer device to evaluate monitored inhalation activity of the user and select the different usage tier based on whether the monitored inhalation activity satisfies a predetermined tier-transition criterion.

15. The computer-implemented method of claim 14, wherein the tier-transition criterion comprises determining a cumulative inhalation count over a predetermined time interval.

16. The computer-implemented method of claim 14, wherein the tier-transition criterion comprises evaluating time intervals between successive inhalation events and determining whether to update a usage tier based on the evaluated time intervals.

17. The computer-implemented method of claim 14, wherein the tier-transition criterion comprises measuring a duration of non-use of the vaporizer device and determining whether to update a usage tier based on the measured duration.

18. A computer program product (CPP) comprising a program of instructions tangibly embodied on a non-transitory computer readable medium wherein, when the instructions are executed on a processor, the processor causes operations to be performed to manage operation of a vaporizer device, the operations comprising:
monitor inhalation events of a user;
determine, for a current usage tier, whether the monitored inhalation events exceed an inhalation limit of the current usage tier within a corresponding time period;
upon determining that the inhalation limit is exceeded, enforce a lockout duration period by disabling an atomizer of the vaporizer device for a predetermined lockout duration defined by the current usage tier such that the user is locked out from using the vaporizer device during the lockout; and
update the current usage tier to a different usage tier of a plurality of usage tiers by executing tier-transition operations, the tier-transition operations causing a logic board of the vaporizer device to evaluate monitored inhalation activity of the user and select the different usage tier based on whether the monitored inhalation activity satisfies a predetermined tier-transition criterion.

19. The computer program product of claim 18, wherein monitoring inhalation events comprises evaluating inhalation events within a rolling time window defined as a continuously shifting interval that extends backward from a current moment and includes only inhalation events occurring within the interval, such that inhalation events occurring prior to the interval are excluded as time advances.

20. The computer program product of claim 18, wherein the operations further comprise:
maintain one-way progression through the plurality of usage tiers such that, after the current usage tier is updated to a lower-allowance usage tier, prevent updating the current usage tier to a higher-allowance usage tier.
